(19) United States
(12) Patent Application Publication
(10) Pub. No.: US 2019/0099289 A1
(43) Pub. Date: Apr. 4, 2019

(54) Breastfeeding Therapeutic Hot/Cold Pack

(71) Applicant: Nicole Larken Beck, (US)

(72) Inventor: Nicole Larken Beck, Dallas, TX (US)

(73) Assignee: Nicole Beck, Dallas, TX (US)

(21) Appl. No.: 16/150,272

(22) Filed: Oct. 3, 2018

Related U.S. Application Data
(60) Provisional application No. 62/566,740, filed on Oct. 2, 2017.

(51) Int. Cl. A61F 7/02; A61M 21/02

(52) U.S. Cl. CPC A61F 7/02; A61F 2007/0021; A61M 21/02

(57) ABSTRACT

The present invention is an improved therapeutic breast pack that helps enhance the breastfeeding experience. The invention provides the user with moist heat long enough to provide therapy to both breasts individually, before nursing and/or pumping. The present invention is filled with dried lavender buds in addition to the whole flaxseed. The flaxseed provides the moist heat (when warmed in microwave), and the dried lavender buds provides relaxation aromatherapy to the mother and baby. Relaxation can help the mother release milk easier, and can help the baby soothe and latch easier. The product is designed so the middle is open to protect the mother's nipple while providing therapy to her breast. Finally, the present invention has a microwavable, removable and machine washable cover. This allows for easy cleaning and usage. The cover does not need to be removed when warming the product in the microwave.

### CROSS-REFERENCE

This Utility Patent claims the benefit of provisional patent application No. 62/566,740.

### BACKGROUND OF INVENTION

I had many breastfeeding challenges with my first child. My breasts were full of milk, but they wouldn't let it all down. I also had many clogged milk ducts. My OBGYN told me to apply moist heat to my breasts by applying a warm, wet washcloth before nursing or by taking a warm shower. Both of these remedies worked, but were cumbersome. There are a few products on the market provided an inadequate amount of moist heat. I needed to nurse immediately after applying therapy for the most effective results. I didn't have time to walk to the microwave and re-warm it, then apply to the second breast. If I waited to warm it after nursing on the first breast, my baby became upset before I could warm it back up and apply therapy on the second breast. Many times during breastfeeding my nipples were sore and sensitive. I wanted a product that didn't touch or apply heat to them. I also wanted something I could remove the cover and wash if needed. Lastly, being a first time mom, I needed help relieving stress and relaxing. My doctor said if I stress, the baby would stress. This hinders milk flow and creates latching problems. That is how I discovered the creation of Milkease. I wanted a natural, painless, quick and easy solution. Whole flaxseed has high oil content and extracts oil when warmed. This invention uses enough flaxseed to provide moist heat therapy to both breasts, individually, without re-warming it. It also contains dried lavender buds to provide much needed aromatherapy to the mother and baby. The aromatherapy aids in relaxation and helps provide a more effective breastfeeding experience. It has a cover that is microwavable, removable and washable. The cover does not need to be removed before warming in the microwave. Washing the cover in the washing machine is convenient when it gets dirty. Lastly, the product has an open middle area, which protects the nipple during usage while providing therapy to the breast.

Prior art exist, but have some combination of the following limitations:
Uses other seeds and grains in addition to flaxseed in the insert. Flaxseed has high oil content and produces moist heat when heated. The moist heat is what helps open the milk ducts, release clogged milk ducts and increase milk flow. Other seeds and grains with low oil content would provide sub-optimal results.
Doesn't provide aromatherapy.
The design doesn't allow for the nipple to remain untouched and protected while providing therapy to the breast.
Does not have a removable and washable cover.
Does not provide the amount of flaxseed to provide therapy to both breasts before re-warming.

### SUMMARY

The present invention is a natural, breastfeeding therapy pack that consists of a pillow filled with whole flaxseed (provides moist heat) and dried lavender buds. It contains enough flaxseed to provide moist heat to both breasts before needing to be re-warmed in the microwave. The dried lavender buds provide aromatherapy that aids in relaxation for the mother and baby. This filled pillow has a cover that is microwavable, removable and washable. This makes usage easy, as the cover does not need to be removed while warming in the microwave. It also makes it easy to clean if needed. Lastly, the invention allows the breast to receive therapy while the nipple remains untouched and protected from the heat. Nipples become soar and sensitive during breastfeeding, and heat can be painful.

### BRIEF DESCRIPTION OF THE DRAWINGS

FIG. 1: The insert (element 3) that is filled with whole flaxseeds (element 2) and dried lavender buds (element 1).

FIG. 2: The back of the cover (element 4) with the hook-and-loop fastener open (element 5) so you can see the insert inside (element 3). The opening (element 6) that keeps the nipple untouched and protected from the heat.

FIG. 3: The invention from the top view facing down.

FIG. 4: The invention from the bottom view, facing up (toward the ‘legs’ of the invention).

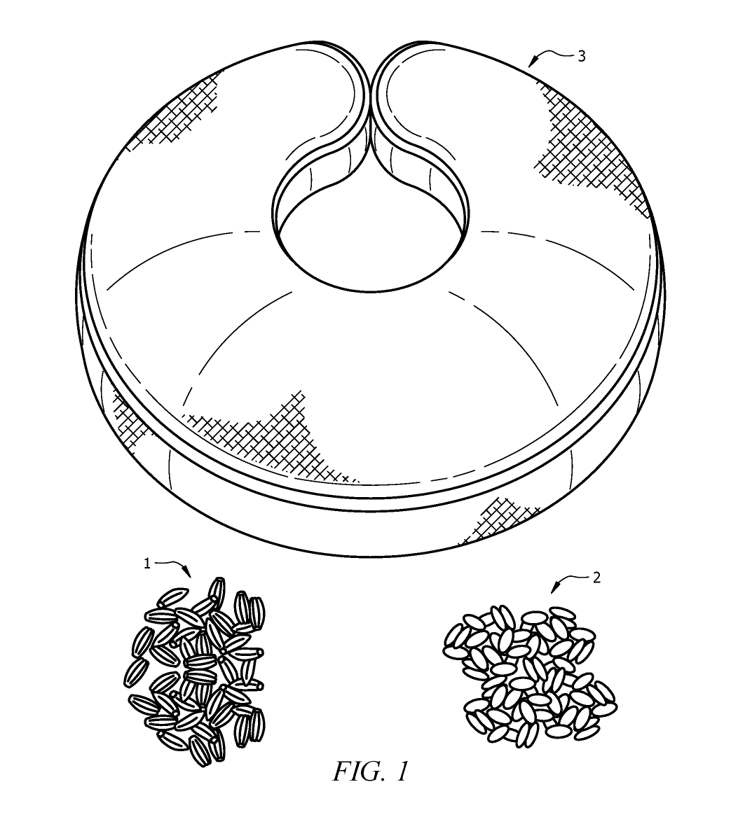

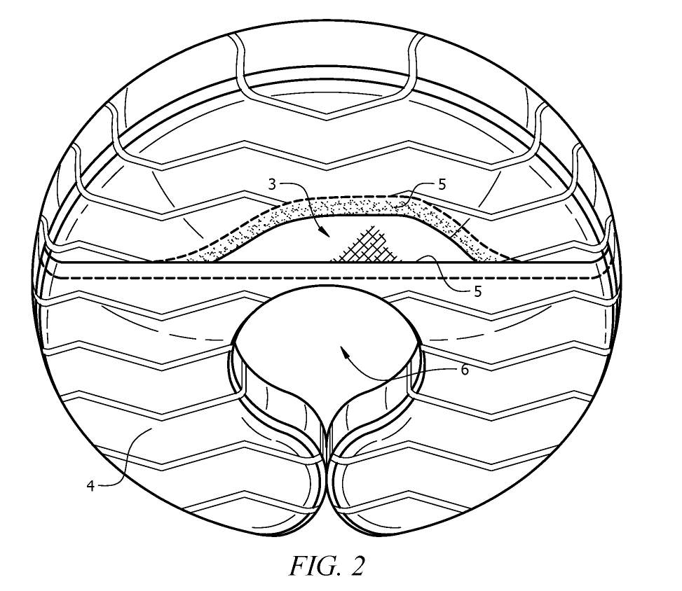

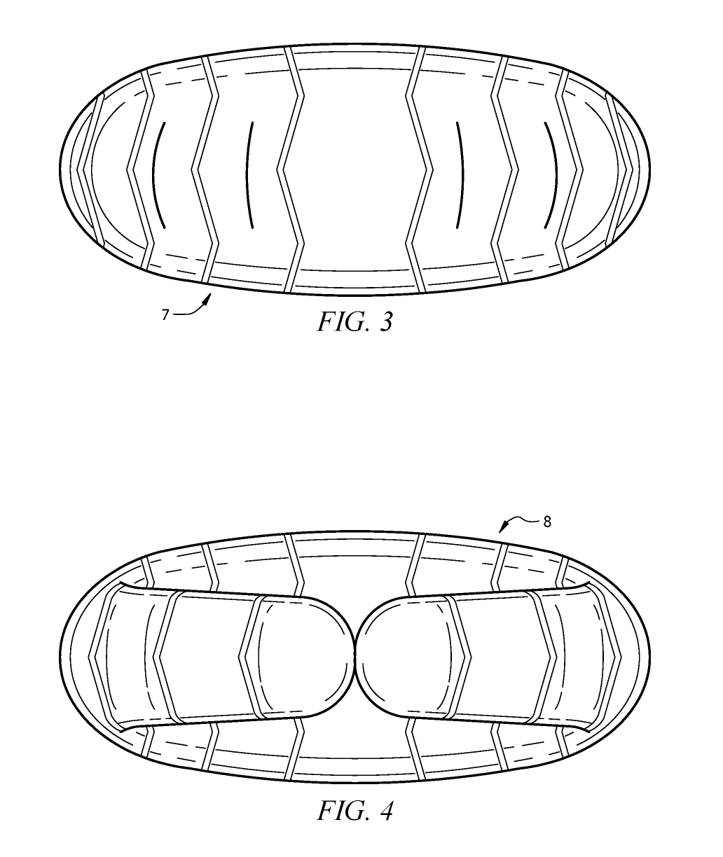

### DETAILED DESCRIPTION

The product makeup,
The product is created by combining two pieces—the insert (element 3) & the outer cover (element 4). The insert (element 3) is a pillow made out of (but is not limited to) 100% cotton. This pillow is filled with whole flaxseed (element 2) and dried lavender buds (element 1). A cover (element 4) made of (but is not limited to) 100% cotton is placed around the insert and is enclosed with (but is not limited to) a microwavable hook-and-loop fastener (element 5) on the backside. The product is assembled when purchased.

The embodiment,
The user places the product in the microwave for 40 seconds (increasing by 10 second intervals until desired temperature). The heat from the microwave allows the whole flaxseed to extract oil and create moist heat.
The user applies the warm product to one of their breasts (or affected area(s)) for 1-5 minutes.
Once therapy is complete on the first breast, the product is applied to the second breast for therapy.
The user is to nurse and/or pump on the first breast immediately after removing the product. The second breast receives therapy, as the first breast is being nursed and/or pumped.

1: A breastfeeding apparatus for providing moist heat therapy, comprising:
An insert filled with enough whole flaxseed to provide therapy on both breasts individually, without being re-warmed; and
A second filling of dried lavender buds to provide aromatherapy; and
A cover that is microwavable, removable and washable; and
An opening in the middle of the product to keep the nipple untouched and protected from heat.

2: A breastfeeding apparatus for providing moist heat therapy, comprising:
An insert filled with enough whole flaxseed to provide therapy on both breasts individually, without being re-warmed; and
A cover that is microwavable, removable and washable; and
An opening in the middle of the product to keep the nipple untouched and protected from heat.
